(19) United States
(12) Patent Application Publication
(10) Pub. No.: US 2026/0174495 A1
(43) Pub. Date: Jun. 25, 2026

(54) ELECTROMAGNETIC COLPOTOMY CUP SYSTEM

(71) Applicant: The Medical College of Wisconsin, Inc., Milwaukee, WI (US)

(72) Inventor: Hadi Omar Saleh, Middleton, WI (US)

(21) Appl. No.: 19/128,963

(22) PCT Filed: Nov. 9, 2023
(86) PCT No.: PCT/US2023/079213

§ 371 Date: May 9, 2025

Related U.S. Application Data
(60) Provisional application No. 63/383,308, filed on Nov. 11, 2022.

(51) Int. Cl. A61B 18/14; A61B 18/20

(52) U.S. Cl. CPC A61B 18/1482; A61B 18/20; A61B 2018/141

(57) ABSTRACT

A colpotomy system includes a colpotomy cup, an electromagnet that is coupled to the colpotomy cup (thereby forming an electromagnetic colpotomy cup), and a bipolar electrocautery wand configured for use with the colpotomy cup and electromagnet. The electromagnet is used to generate an inwardly directed force, known as the Lorentz force, to direct the electrocautery wand towards the center of the colpotomy cup. This allows the physician to direct the wand towards the center of the uterine axis and away from the bladder as well as other surrounding structures in the pelvic cavity.

### BACKGROUND

Colpotomy is one of the final steps in a hysterectomy procedure. During the colpotomy step, a circular incision is made in the vaginal tissue to separate the uterus from the vagina. The incision can be made with a scalpel or with an electrocautery tool and with the aid of a uterine manipulator to position the vagina and the cervix to facilitate separation.

In robotically assisted and non-assisted laparoscopic hysterectomies, one of the difficulties of the procedure is to avoid inadvertent damage to the bladder during uterine excision via electrocautery. Typically, a colpotomy cup, which contains circumferential ridges, is used to help guide the physician when excising the uterus. Use of the colpotomy cup still requires the skill of the physician to prevent inadvertent damage to the bladder during excision. There remains a need, then, to provide a tool that facilitates safer excision during laparoscopic hysterectomies to prevent the long-term repercussions on quality of life that may happen with inadvertent damage to the bladder.

### SUMMARY OF THE DISCLOSURE

The present disclosure addresses the aforementioned drawbacks by providing a colpotomy system that includes a colpotomy cup and an electromagnet coupled to the colpotomy cup. The colpotomy cup has a top portion of a first diameter and a base portion of a second diameter that is smaller than the first diameter. The base portion has a hole formed therein, where the hole circumscribes a longitudinal axis extending from the base portion to the top portion of the colpotomy cup. The electromagnet is coupled to at least one of the top portion or the base portion of the colpotomy cup. When a current passes through the electromagnet, a magnetic field is generated along the longitudinal axis of the colpotomy cup thereby generating a Lorentz force directed inward towards the longitudinal axis.

It is another aspect of the present disclosure to provide a colpotomy kit that includes a colpotomy cup, a solenoid coupled to the colpotomy cup, and a bipolar electrocautery wand. The colpotomy cup has atop portion of a first diameter and a base portion of a second diameter that is smaller than the first diameter. The base portion has a hole formed therein, where the hole circumscribes a longitudinal axis extending from the base portion to the top portion. The solenoid is coupled to at least one of the top portion or the base portion of the colpotomy cup. The bipolar electrocautery wand has an electrical lead portion extending along a first direction and a cutting edge portion extending along a second direction that is perpendicular to the first direction. When a current passes through the solenoid, a magnetic field is generated along the longitudinal axis of the colpotomy cup thereby generating a Lorentz force directed inward towards the longitudinal axis of the colpotomy cup. Additionally, when a current passes through the cutting edge portion of the electrocautery wand, the Lorentz force pushes the cutting edge portion of the electrocautery wand towards the longitudinal axis of the colpotomy cup.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 illustrates an example colpotomy system in accordance with some embodiments described in the present disclosure, which includes a colpotomy cup, an electromagnet coupled to a surface of the colpotomy cup, and an electrocautery wand that is configured to be forced towards the central axis of the colpotomy cup by a Lorentz force generated by the magnetic field of the electromagnet interacting with the current flowing in the electrocautery wand.

FIGS. 2A-2D illustrate different configurations for coupling the electromagnet to the colpotomy cup.

FIGS. 3A-3C illustrate an example of an electromagnet constructed as a nested pair of solenoids, which may be implemented in some embodiments of the present disclosure.

FIGS. 4A-4C illustrate example magnetic fields generated by supplying current to a nested pair of solenoids, such as the nested pair of solenoids illustrated in FIGS. 3A-3C.

FIG. 5 is a Lorentz force diagram showing Lorentz forces generated by an outer, ring magnetic field (F_{ring}) and an inner magnetic field (Fᵢₙ) of a nested pair of solenoids.

FIG. 6 is an example circuit diagram for a solenoid electromagnet that can be used in some embodiments of the colpotomy system described in the present disclosure.

FIG. 7 is an example circuit diagram for a bipolar electrocautery wand that can be used in some embodiments of the colpotomy system described in the present disclosure.

### DETAILED DESCRIPTION

Described here is a colpotomy system or kit, which generally includes a colpotomy cup, an electromagnet that is coupled to the colpotomy cup (thereby forming an electromagnetic colpotomy cup), and an electrocautery wand configured for use with the colpotomy cup and electromagnet. The electromagnetic colpotomy cup improves procedure safety by using the electromagnet to generate an inwardly directed force, known as the Lorentz force, which can be used to direct the electrocautery wand towards the center of the colpotomy cup. This allows the physician to direct the wand towards the center of the uterine axis and away from the bladder as well as other surrounding structures in the pelvic cavity. Advantageously, the colpotomy systems described in the present disclosure can improve patient safety for both robotically assisted and non-robotically assisted laparoscopic hysterectomy procedures.

An electromagnetic setup, instead of a permanent magnet setup, allows the physician to turn the magnetic field on and off when it is needed. This is important because a permanent magnetic field could interfere with the initial stages of the procedure when the physician is maneuvering within the pelvic cavity.

As described below in more detail, the electromagnetic colpotomy cup and electrocautery wand are constructed such that the directions of the magnetic field generated by the electromagnet and the current passing through the electrocautery wand are perpendicular, such that a force on the electrocautery wand will be generated towards the center of the electromagnet.

Normally, during a laparoscopic hysterectomy, a unipolar circuit is used in the electrocautery wand to heat and cauterize the tissue. It is an advantage of the systems and methods described in the present disclosure that a bipolar circuit is used in the electrocautery wand, which facilitates generation of the Lorentz force along the cutting edge of the wand.

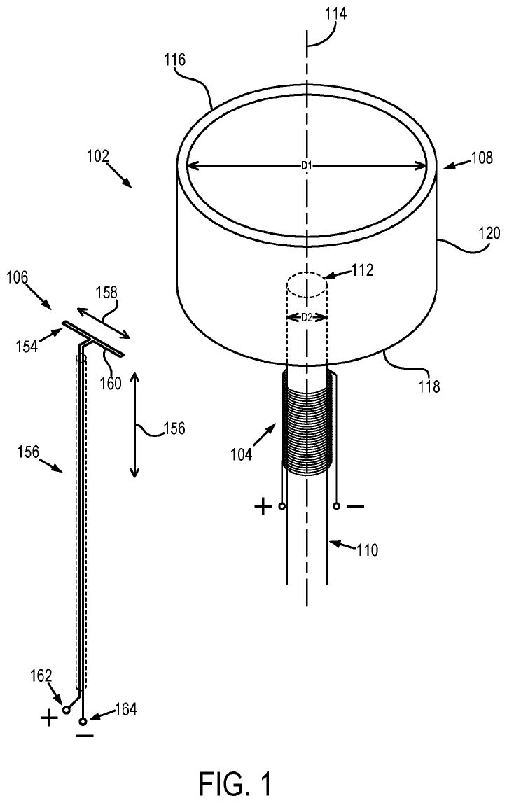

Referring now to FIG. 1, an example colpotomy system, or kit, is shown. As described above, the colpotomy system generally includes a colpotomy cup 102, an electromagnet 104, and an electrocautery wand 106.

The colpotomy cup 102 generally includes a top portion 108 and a base portion 110. The base portion 110 has a hole 112 formed therein to provide access for surgical tools during a procedure and/or for coupling the colpotomy cup 102 to a surgical instrument, such as a uterine manipulator. The hole 112 circumscribes a longitudinal axis 114 that extends from the base portion 110 to the top portion 108 of the colpotomy cup 102. The top portion 108 of the colpotomy cup 102 has a generally circular cross-section with a first diameter, D1. The base portion 110 of the colpotomy cup 102 also has a generally circular cross-section with a second diameter, D2, that is smaller than the first diameter, D1. As a non-limiting example, the first diameter may be selected from the range of 3-4 cm, such as 3 cm, 3.5 cm, or 4 cm.

The top portion 108 of the colpotomy cup 102 generally extends along the longitudinal axis from a first surface 116 to a second surface 118. The first surface 116 may include a leading edge of the colpotomy cup 102, which comes into contact with the vaginal fornix during use. The second surface 118 of the colpotomy cup 102 couples to the base portion 110 of the colpotomy cup 102. A sidewall 120 extends between the first surface 116 and the second surface 118. In some embodiments, one or more openings may be formed in the sidewall 120 to facilitate access to the inner volume of the top portion 108 and/or to facilitate visualization of tissues during a surgical procedure.

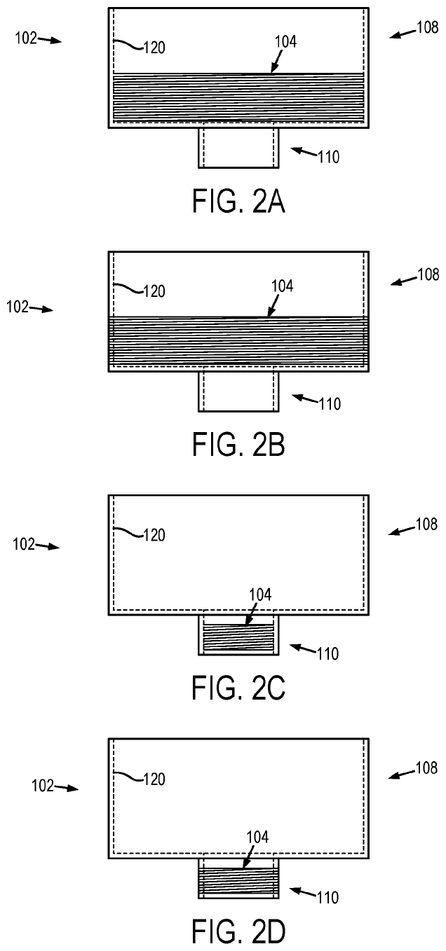

In general, the electromagnet 104 can be constructed as a solenoid. In some embodiments, the electromagnet 104 is designed to have a circular cross-section (i.e., be constructed as circular conductive loops circumscribing the longitudinal axis 114). The electromagnet 104 is coupled to a surface of the colpotomy cup 102, and in different embodiments may be coupled to different surfaces of the colpotomy cup 102, as shown in FIGS. 2A-2D. For example, in FIG. 2A, the electromagnet 104 is shown as being coupled to the inner surface of the sidewall 120 of the top portion 108 of the colpotomy cup 102. In FIG. 2B, the electromagnet 104 is shown as being coupled to the outer surface of the sidewall 120 of the top portion 108 of the colpotomy cup 102. Additionally or alternatively, as shown in FIG. 2C, the electromagnet 104 can be coupled to the inner surface of the base portion 110 of the colpotomy cup 102. In FIG. 2D, the electromagnet 104 is shown as being coupled to the outer surface of the base portion 110 of the colpotomy cup 102.

The electrocautery wand 106 is composed of an electrical lead portion 152 and a cutting edge portion 154. In general, the electrical lead portion 152 extends in or along a first direction 156 and the cutting edge portion 154 extends in or along a second direction 158 that is perpendicular to the first direction 156. Thus, in some embodiments, the electrocautery wand 106 can include a T-shaped electrode that include the electrical lead portion 152 and the cutting edge portion 154. The electrical lead portion 152 and cutting edge portion 154 may be composed of a single conductive element 160. For example, the conductive element 160 may begin at a starting point 162 in the electrical lead portion 152, then extend along the first direction 156 in the electrical lead portion 152 before turning to extend along the second direction 158 in the cutting edge portion 154. The conductive element 160 may then turn back on itself and extend in the opposite direction along the second direction 158 before returning back towards the electrical lead portion 152 along the second direction 158. The conductive element 160 may then turn and extend back along the first direction 156 to an ending point 164 in the electrical lead portion 152.

The electrocautery wand 106 is preferably configured as a bipolar electrocautery wand and composed of a high-resistance wire. When a current is applied through the conductive element 160, a Lorentz force will be generated along the cutting edge portion 154 of the electrocautery wand 106, which directs the electrocautery wand 106 towards the longitudinal axis 114.

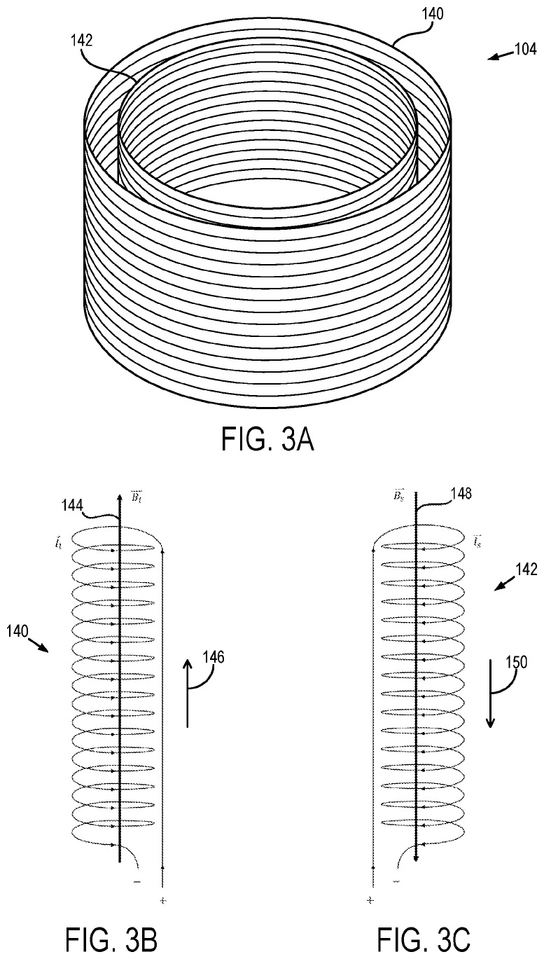

In the illustrated examples of FIGS. 1 and 2A-2D, the electromagnet 104 is shown as a solenoid. In some alternative examples, the electromagnet 104 may be implemented as a pair of nested solenoids, as illustrated in FIGS. 3A-3C. For example, as illustrated in FIG. 3A, the electromagnet 104 may include a first solenoid 140 and a second solenoid 142 that is nested within the first solenoid 140. The first solenoid 140 has a larger diameter than the second solenoid 142 such that the second solenoid 142 can be arranged within the inner volume of the first solenoid 140 and, therefore, circumscribed by the first solenoid 140. As shown in FIG. 3B, the first solenoid 140 may be constructed such that it generates a first magnetic field 144 oriented along a first direction 146, and as shown in FIG. 3C, the second solenoid 142 may be constructed such that it generates a second magnetic field 148 oriented along a second direction 150 that is different than the first direction 146. For example, the first direction 146 and the second direction 150 may be opposite directions (e.g., a +z direction and a −z direction).

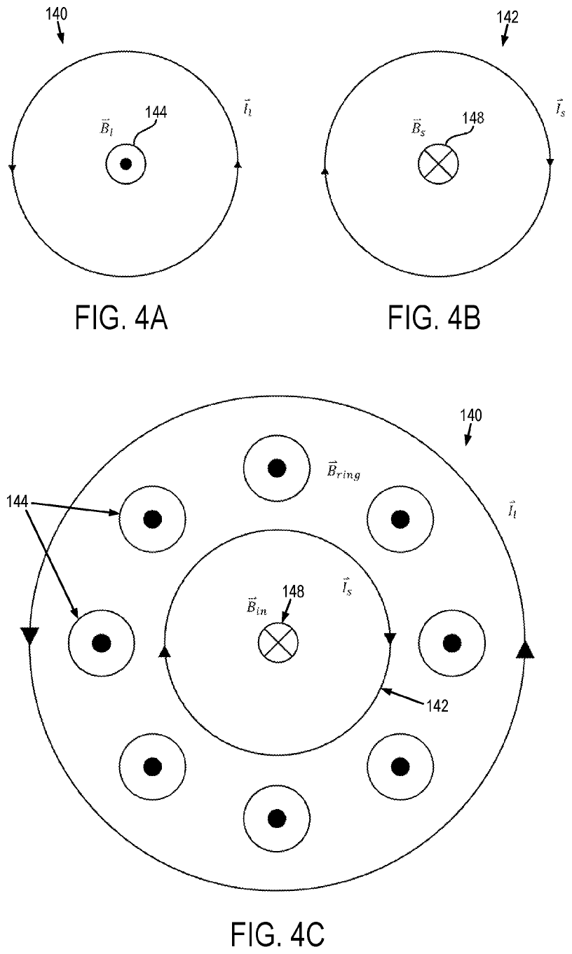

An example of the current flowing in the first solenoid 140 is shown in FIG. 4A and an example of the current flowing in the second solenoid 142 is shown in FIG. 4B. As shown in FIG. 4A, the current flowing in the first solenoid 140 generates a first magnetic field 144, \vec{B}ₗ, along a first direction 146 that is oriented out of the page. As shown in FIG. 4B, the current flowing in the second solenoid 142 generates a second magnetic field 148, \vec{B}ₛ, along a second direction 150 that is oriented into the page. When the first solenoid 140 and the second solenoid 142 are nested together (e.g., as shown in FIG. 3A), a “ring” magnetic field can be generated, as illustrated in FIG. 4C. In these instances, the magnetic field in the center of the second solenoid 142 is oriented along the second direction 150 while the magnetic field in the space between the outer surface of the second solenoid 142 and the inner surface of the first solenoid 140 is oriented along the first direction 146. As illustrated, the ring magnetic field, \vec{B}_{ring}, may be pointing out of the page and may be defined as \vec{B}_{ring}=\vec{B}ₗŷ, whereas the inner magnetic field, \vec{B}ᵢₙ may be pointing into the page and may be defined as \vec{B}ᵢₙ=(\vec{B}ₗ−\vec{B}ₛ)ŷ.

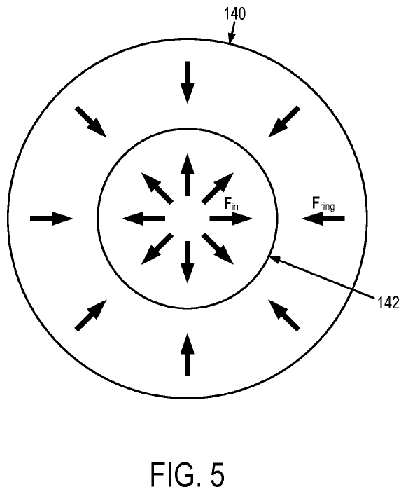

In some implementations, the current supplied to the second solenoid 142 (i.e., the inner solenoid) can be adjusted such that the second magnetic field 148 is equal to zero. One advantage of this nested solenoid setup is that the outer magnetic field ring (e.g., the first magnetic field 144 generated in the space between the outer surface of the second solenoid 142 and the inner surface of the first solenoid 140) can be used to attract the cutting tool (e.g., electrocautery wand 106), whereas the second magnetic field 148 (i.e., the inner, downwards magnetic field) repels the cutting tool (e.g., electrocautery wand 106). This combination of attraction and repulsion facilitates cutting across a given distance through the cervix. FIG. 5 illustrates a Lorentz force diagram representing the direction of the force exerted on the cutting tool (e.g., electrocautery wand 106) when using a nested solenoid setup for the electromagnet 104. The force diagram illustrates the attraction-repulsion described above.

As another advantage, the nested solenoid setup of the electromagnet 104 prevents unobstructed movement of the cutting tool (e.g., electrocautery wand 106) through the cervical tissue. For example, the repulsive core magnetic field (e.g., second magnetic field 148) decreases the risk of the cutting tool (e.g., electrocautery wand 106) moving excessively through tissue, which could otherwise damage neighboring structures, such as the bladder.

As a non-limiting example, the electromagnet is comprised of a solenoid, which is connected to a power-source in a series circuit configuration. Given the circular shape of the vaginal fornix and cervix, a solenoid composed of circular coils was chosen as the element within the proposed electrical circuit. When a constant direct current (“DC”) is passed is passed through the circuit, the solenoid will generate a constant magnetic field, per Ampere's law.

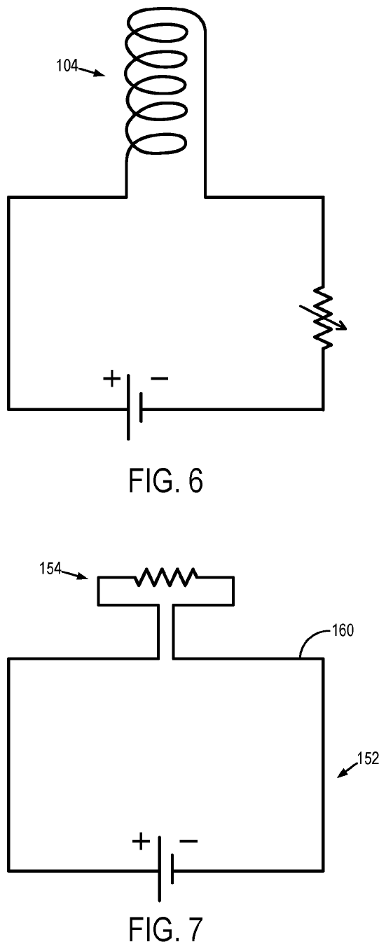

In some embodiments, a variable resistor can be used to vary the magnitude of current within the circuit, as shown in FIG. 6, which in turn allows for the manipulation of magnetic field strength generated by the solenoid. Recall that the magnitude of the magnetic field by a solenoid is described by:

$$\vert B \vert = {μ}_{0} {I}_{S} N$$

where |B| is the magnitude of the magnetic field, B; μ₀ is the magnetic permeability of free space in a vacuum; I_{S} is the magnitude of the current passing through the solenoid; and N is the number of turns within the solenoid. In some implementations, the interior of the solenoid is assumed to be free space. In other configurations, however, a ferromagnetic core can be placed within the inner volume of the solenoid to augment the magnetic field, in which case, μ₀ would be replaced by the corresponding value for the magnetic permeability, μ, of the material used (i.e., where μ>μ₀).

Given that the magnitude of the Lorentz force acting on the electrocautery wand is directly proportional to magnitude of the magnetic field, changes in the variable resistor allows the physician to control the inwards force acting on the wand during excision.

It is relevant to consider the amount of heat produced by the solenoid during operation of the colpotomy system. This is because excessive solenoid heating may lead to collateral damage to vaginal structures, given its placement near the cervix and vaginal vault.

Note that the power consumption of a circuit is described by:

$$P = IV or P = \frac{{V}^{2}}{R} or P = {I}^{2} R ;$$

where P is the energy consumed per unit time, I is the current passing through the circuit, and V is the potential difference across the circuit.

In some configurations, it can be assumed that the majority of the energy lost or dissipated by the circuit is through heat (i.e., other forms of energy loss are negligible). This allows the work done by the circuit to be equated to the energy lost due to heat:

$${W}_{D} = Q$$

where W_{D} is the energy consumed by the circuit and Q is the energy released as heat by the circuit.

In some configurations, it can be assumed that the current, I, and the potential difference, V, within the circuit are constant with respect to time, during steady state. In these instances, and given that:

$$\frac{d {W}_{D}}{d t} = P or \frac{d {W}_{D}}{d t} = IV or \frac{d {W}_{D}}{d t} = \frac{{V}^{2}}{R} ;$$

this first order ordinary differential equation to arrive at:

$${W}_{D} = ( \frac{{V}^{2}}{R} ) t or {W}_{D} = {I}^{2} Rt .$$

Thus, the heat produced by the whole circuit can be described as:

$$Q = ( \frac{{V}^{2}}{R} ) t or Q = {I}^{2} Rt .$$

Note that to consider the amount of heat generated by the solenoid alone, the current passing across the solenoid, at steady state, is considered alone, such that:

$${Q}_{sol} = {I}^{2} {R}_{sol} t .$$

Based on FIG. 6, the solenoid and variable resistor are in a series configuration and, therefore, the current passing through the circuit can be found by applying Kirchhoff's law for the single loop around the circuit:

$$V - I {\sum }_{{i = 1}^{}}^{N} {R}_{i} = 0 ;$$

where Rᵢ represents the ith resistant element within the circuit.

In some configurations, it can be assumed that the wires between circuit components are short and are composed of a material with low resistivity (e.g., copper), such that their resistance is negligible.

Given that there are only two resistance elements within the example circuit, namely the solenoid and the variable resistor, the current passing through the circuit, at steady state, can be described by:

$$I = \frac{{V}_{source}}{{R}_{sol} + {R}_{v ar}} .$$

Substituting this value for current, the heat produced by the solenoid is:

$${Q}_{sol} = {( \frac{{V}_{source}}{{R}_{sol} + {R}_{v ar}} )}^{2} {R}_{sol} t$$

Where V_{source} represents the potential difference across the circuit, delivered by a DC power source; Rₛₒₗ is the resistance of the solenoid; Rᵥₐᵣ is the resistance of the variable resistor; and t is the time elapsed since completing the circuit (i.e. turning it “ON”).

To determine the changes in temperature of the solenoid as a function of time, the following can be considered:

$${Q}_{sol} = {m}_{sol} {c}_{wire} ( {T}_{f} - {T}_{i} )$$

where mₛₒₗ is the mass of the solenoid; c_{wire} is the specific heat capacity of the wire material used; T_{f} is the final temperature of the solenoid, after some time “t” has elapsed; and T the initial temperature of the solenoid, prior to completing the circuit.

Thus, equating these expressions for Qₛₒₗ, the following expression can be arrived at:

$${m}_{sol} {c}_{wire} ( {T}_{f} - {T}_{i} ) = {( \frac{{V}_{source}}{{R}_{sol} + {R}_{v ar}} )}^{2} {R}_{sol} t .$$

Thus, the function describing the final temperature of the solenoid is:

$${T}_{f} = ( \frac{1}{{m}_{sol} {c}_{wire}} {( \frac{{V}_{source}}{{R}_{sol} + {R}_{v ar}} )}^{2} {R}_{sol} ) t + {T}_{i} .$$

It is noted that the resistance of the solenoid, Rₛₒₗ, can be expressed as a function of the length, cross-sectional area, and resistivity of the material used to form the solenoid. Recalling that:

$$R = \frac{{ρ}_{res} L}{A}$$

where ρᵣₑₛ is the resistivity of the material used; L is the length of the wire used; and A is the cross-sectional area of the wire.

Given a helical shape for the solenoid, its resistance can be expressed as:

$${R}_{sol} = \frac{4 {ρ}_{res} N \sqrt{{( π {D}_{sol} )}^{2} + {( \frac{{H}_{sol}}{N} )}^{2}}}{{π ( {D}_{wire} )}^{2}} ;$$

where ρᵣₑₛ is the resistivity of the material used; N is the number of turns within the solenoid; Dₛₒₗ is the diameter of the solenoid, which in a preferred embodiment is sized such that the solenoid can be coupled to a colpotomy cup (e.g., within the top portion of the cup, circumscribing the exterior of the top portion of the cup, within the base portion of the cup, circumscribing the exterior of the base portion of the cup), which in some instances may be a standard colpotomy cup; Hₛₒₗ is the height of the solenoid, which in a preferred embodiment is sized such that the solenoid can be coupled to a surface of the colpotomy cup, which in some instances may be a standard colpotomy cup; and D_{wire} is the diameter of the wire used to form the solenoid.

It is noted that the mass of the solenoid, mₛₒₗ, can be expressed as a function of the length, cross-sectional area, and resistivity of the material used to form the solenoid. For instance:

$$m = {ρ}_{den} A L ;$$

where ρ_{den} is the density of the material used; A is the cross-sectional area of the wire; and L is the length of the wire used.

Given a helical shape for the solenoid, its mass can be expressed as:

$${m}_{sol} = \frac{{ρ}_{den} {π ( {D}_{wire} )}^{2}}{4} ( N \sqrt{{( π {D}_{sol} )}^{2} + {( \frac{{H}_{sol}}{N} )}^{2}} ) ;$$

where ρ_{den} is the density of the material used; N is the number of turns within the solenoid; Dₛₒₗ is the diameter of the solenoid, which in a preferred embodiment is sized such that the solenoid can be coupled to a colpotomy cup (e.g., within the top portion of the cup, circumscribing the exterior of the top portion of the cup, within the base portion of the cup, circumscribing the exterior of the base portion of the cup), which in some instances may be a standard colpotomy cup; Hₛₒₗ is the height of the solenoid, which in a preferred embodiment is sized such that the solenoid can be coupled to a surface of the colpotomy cup, which in some instances may be a standard colpotomy cup; and D_{wire} is the diameter of the wire used to form the solenoid.

Newton's law of cooling states that the rate heat transfer between two objects is directly proportional to the differences in their temperatures. Given Newton's law of cooling is most closely obeyed in purely conduction types of cooling, its application is appropriate to model changes in temperature within cervical tissue. This is because there are minimal quantities of fluid and air present within the vaginal canal and surrounding tissues near the cervix; thus, heat loss via convection is minimal.

It can therefore be assumed that, although radiative heat loss occurs, this type of heat loss is likely negligible when compared to conduction, based on application of the Stephan-Boltzmann Law for black body radiation. The following factors contribute to the minimal amount of radiative heat loss: low temperature difference between the device during operation peak (approximately 40° C.) and surrounding tissues (approximately 37° C.); and the small surface area of the solenoid.

The estimated rate of heat transfer via radiative heat loss during operation of the device can be given by:

$${( \frac{d Q}{d t} )}_{Radiation} = σ e A ( {( {T}_{sol} )}^{4} - {( {T}_{cervix} )}^{4} ) .$$

Substituting 5.67×10⁻⁸ Wm⁻²K⁻⁴, Tₛₒₗ=313 K, T_{cervix}=310 K, and e=1, the following expression can be derived:

$${( \frac{d Q}{d t} )}_{Radiation} = 20.6 A ;$$

where A is the surface area of the solenoid.

The estimated rate of heat transfer via conductive heat loss during operation of the device can be given by:

$${( \frac{dQ}{d t} )}_{Conduction} = h A ( {T}_{sol} - {T}_{cervix} ) .$$

Substituting Tₛₒₗ=313 K and T_{cervix}=310 K, the following expression can be derived:

$${( \frac{d Q}{d t} )}_{Conduction} = 3 hA ;$$

where A is the heat transfer surface area and h is the heat transfer coefficient.

Given that the thermal conductivity coefficient for copper is 386 Wm⁻¹K⁻¹ at 20° C. under 1 atm of pressure, a large value for the heat transfer coefficient, h, is expected, such that the rate of heat loss via conduction is very large relative to that of radiative heat loss.

Using a simplified formulation for Newton's law of cooling, the following expression can be arrived at:

$$\frac{d {T}_{cervix}}{d t} = k ( {T}_{sol} - {T}_{cervix} ) .$$

Recalling that Tₛₒₗ is a function that varies with time, and can be described by:

$${T}_{sol , f} = ( \frac{1}{{m}_{sol} {c}_{wire}} {( \frac{{V}_{source}}{{R}_{sol} + {R}_{va r}} )}^{2} {R}_{sol} ) t + {T}_{sol , i} .$$

It can be assumed that, during a procedure, the solenoid will be initially placed near the cervix prior to laparoscopic manipulation of the pelvic cavity. In other words, the solenoid will be placed near the cervix for a prolonged period of time, such that it can be assumed that T_{sol,i}=T_{cervix}(0).

Thus, the function for Tₛₒₗ can be rewritten as:

$${T}_{sol} = ( \frac{1}{{m}_{sol} {c}_{wire}} {( \frac{{V}_{source}}{{R}_{sol} + {R}_{v ar}} )}^{2} {R}_{sol} ) t + {T}_{cervix} ( 0 ) .$$

Thus,

$$\frac{d {T}_{cervix}}{d t} = k ( ( \frac{1}{{m}_{sol} {c}_{wire}} {( \frac{{V}_{source}}{{R}_{sol} + {R}_{v ar}} )}^{2} {R}_{sol} ) t + {T}_{cervix} ( 0 ) - {T}_{cervix} ) .$$

which can be simplified to the following expression:

$${T}_{cervix} = {T}_{cervix} ( 0 ) + j ( t - \frac{1}{k} ) + {Ce}^{- kt} ;$$

$$where j = ( \frac{1}{{m}_{sol} {c}_{wire}} {( \frac{{V}_{source}}{{R}_{sol} + {R}_{v ar}} )}^{2} {R}_{s o l} ) .$$

Values for “C” and “k” can be determined from experimental data.

Thus, the current passing through the wire of the electromagnet will generate heat, which is dependent on the magnitude of the current as well as the resistivity of the wire material. The electromagnet can be designed and operated to prevent or otherwise mitigate excessive solenoid heating, which could otherwise damage surrounding vaginal tissue. For instance, the solenoid current could be reduced, but the number of turns in the solenoid could be increased, thereby reducing heat generation without compromising the strength of the magnetic field. Additionally or alternatively, the solenoid can be composed of a wire material that has less resistivity. An insulating material could also be used to coat the solenoid in order to reduce the rate of heat transfer to surrounding tissues.

Under a single compartment model of heat transfer from the solenoid to the cervical tissues, the amount of heat transferred from the solenoid to the vaginal compartment, as a function of time, can be determined using the following:

$${Q}_{H} = m c ( {T}_{G} - {T}_{G , i} ) ;$$

where Q_{H} represents the amount of heat transferred as a function of time, m represents the mass of air stored within the vaginal compartment, c represents the specific heat capacity of air, T_{G} represents the temperature of the vaginal compartment as a function of time, and T_{G,i} represents the initial temperature within the vaginal compartment.

Because T_{G,i} is a constant, taking the derivative of the function Q_{H} with respect to time yields:

$$\frac{d {Q}_{H}}{d t} = m c ( \frac{d {T}_{G}}{d t} ) .$$

Because the rate of heat transfer from a heating element is analogous to the power output of the solenoid, the following expressions can be defined:

$$\frac{d {Q}_{H}}{d t} = {P}_{H}$$

$${P}_{H} = m c ( \frac{d {T}_{G}}{dt} ) .$$

$$\frac{d {T}_{G}}{d t} = \frac{{P}_{H}}{m c}$$

The expression above is notable since it refers to the contribution of the solenoid to the rate of change in temperature within the vaginal compartment.

Using Newton's law of cooling, the expression above can be combined with the rate of heat loss to the environment to determine a differential equation describing the change in temperature within the vaginal compartment as a function of time, where:

$$\frac{{dT}_{G}}{dt} = - k ( {T}_{G} - {T}_{O} ) + \frac{{P}_{H}}{mc} ,$$

where dT_{G}/dt represents the rate of change in temperature with respect to time, within the vaginal compartment; k is a constant of proportionality that describes the rate of heat loss between the vaginal compartment and the outside environment; T_{O} represents the temperature of the outside environment, with an assumption in some instances that T_{O} may be considered as a constant, since the temperature within the operating theater is typically maintained at a set value via HVAC; and P_{H} represents the power output of the solenoid.

This differential equation can have the following solution:

$${T}_{G} = {T}_{O} + \frac{{P}_{H}}{kmc} + C {e}^{- kt} .$$

During the early stages of the surgery, the manipulation of the vagina causes the air within the vaginal compartment to become continuous with the air of the operating theater. Consequently, it can be assumed in some instances that the start of temperature of the vaginal compartment at t=0 is the same as that of the operating theater; that is,

$${T}_{G} ( 0 ) = {T}_{O} .$$

Using this assumption, the constant of integration, C, noted above can be determined as,

$$C = - \frac{{P}_{H}}{kmc} ,$$

Therefore, the temperature of the vaginal compartment as a function of time may be given by:

$${T}_{G} = {T}_{O} + \frac{{P}_{H}}{kmc} ( 1 - {e}^{- k t} ) .$$

Experimental data may be used to determine a value for the proportionality constant, k. For example, a Lambert W function can be used, where after a given value T_{G} has been recorded after t has elapsed:

$$k = \frac{1}{j} + \frac{1}{t} W ( - \frac{t}{j} {e}^{- \frac{t}{j}} ) ,$$

$$where$$

$$j = \frac{( {T}_{G} - {T}_{O} ) mc}{{P}_{H}} .$$

The bipolar electrocautery wand is composed of a high-resistance wire that is shaped in a “T” configuration and is connected to a power-source in a series circuit configuration, as shown in FIG. 7. When a current is applied through the wand, a Lorentz force will be generated along the cutting edge of the wire, which directs it towards the center of the solenoid. Note that the current passing through the wire serves two purposes: to generate the heat needed to cauterize uterine and cervical tissue, which facilitates excision; and to generate a Lorentz force which acts along the cutting edge of the wire, towards the center of the solenoid.

The Lorentz force generated along the cutting edge of the electrocautery wand is directly proportional to the cross-product of the current passing through the wand and the magnetic field generated by the solenoid, such that:

$${F}_{wand} = ( {I}_{wand} \times {B}_{sol} ) {L}_{wand} ;$$

where F_{wand} is the force vector generated along the cutting edge of the wand, I_{wand} is the current vector passing through the wand, Bₛₒₗ is the magnetic field vector generated by the solenoid, L_{wand} is the length of the cutting edge of the wand.

Given the simple configuration of the electrocautery wand circuit, the magnitude of its current can be described by:

$${I}_{wand} = \frac{{V}_{source}}{{R}_{wand}} .$$

In some embodiments, the wires between circuit components are short and are composed of a material with low resistivity (e.g., copper), such that their resistance is negligible.

Recalling that the resistance of a high resistance wire, which is used as the cutting edge of the electrocautery wand, can be determined as a function of wire length, and cross-sectional area, the following expression can be utilized:

$${R}_{wand} = \frac{{ρ}_{res} {L}_{wand}}{A} .$$

Given wire thickness and length, it can be found that:

$${R}_{wand} = \frac{4 {ρ}_{res} {L}_{wand}}{π {( {D}_{wire} )}^{2}} .$$

Substituting for values of I_{wand} and Bₛₒₗ, the magnitude of the Lorentz force generated along the cutting edge of the electrocautery wand is:

$$\vert {F}_{wand} \vert = \vert {{V}_{source , W} ( \frac{π ( {D}_{wire , W} )}{4 {ρ}_{res , W} {L}_{wand}} )}^{2} \vert * \vert {μ}_{0} {N}_{sol} ( \frac{{V}_{source , S}}{{R}_{sol} + {R}_{var}} ) \vert * sin θ * {L}_{wand} ;$$

where |F_{wand}| is the magnitude of the Lorentz force, V_{source,W} is the potential difference across the electrocautery wand circuit, D_{wire,W} is the diameter of the wire used to form the electrocautery wand, 4ρ_{res,W} is the resistivity of the wire material used to form the electrocautery wand. L_{wand} is the length of the cutting edge of the wand, and sin θ is the sine of the angle between the current and magnetic field vectors.

The heat generated by the wand can be described by:

$${Q}_{wand} = \frac{{( {V}_{source} )}^{2}}{{R}_{wand}} t ;$$

Where V_{source} is the potential difference across the electrocautery circuit, delivered by a DC power source; R_{wand} is the resistance of the electrocautery wand; and t is the time elapsed since completing the circuit (i.e., since turning the circuit ON).

To determine the changes in temperature of the electrocautery wand as a function of time, the following can be used:

$${T}_{f} = ( \frac{1}{{m}_{wand} {c}_{wire}} \cdot \frac{{( {V}_{source} )}^{2}}{{R}_{wand}} ) t + {T}_{i} .$$

The mass of the wand, m_{wand}, can be expressed as:

$${m}_{wand} = \frac{{ρ}_{den} π {( {D}_{wire} )}^{2} {L}_{wand}}{4} ;$$

where ρ_{den} is the density of the material used, D_{wire} is the diameter of the wire used to form the electrocautery wand, and L_{wand} is the length of the cutting edge of the wand.

Similar to the heat transfer between the solenoid and the cervical tissue, the heat transferred from the electrocautery wand to the cervical tissues can be expressed as:

$${T}_{cervix} = {T}_{cervix} ( 0 ) + j ( t - \frac{1}{k} ) + {Ce}^{- kt} ;$$

$$where :$$

$$j = ( \frac{1}{{m}_{wand} {c}_{wire}} \cdot \frac{{( {V}_{s o u r c e} )}^{2}}{{R}_{wand}} ) .$$

In this example, it was assumed that the electrocautery wand is placed near within the pelvic cavity for a prolonged period of time, such that T_{wand,i}=T_{cervix}(0).

The present disclosure has described one or more preferred embodiments, and it should be appreciated that many equivalents, alternatives, variations, and modifications, aside from those expressly stated, are possible and within the scope of the invention.

1. A colpotomy system, comprising:
a colpotomy cup having a top portion of a first diameter and a base portion of a second diameter that is smaller than the first diameter, wherein the base portion has a hole formed therein, wherein the hole circumscribes a longitudinal axis extending from the base portion to the top portion; and
an electromagnet coupled to at least one of the top portion or the base portion of the colpotomy cup, wherein when a current passes through the electromagnet a magnetic field is generated along the longitudinal axis of the colpotomy cup thereby generating a Lorentz force directed inward towards the longitudinal axis.

2. The colpotomy system of claim 1, wherein the electromagnet circumscribes an exterior surface of the base portion of the colpotomy cup.

3. The colpotomy system of claim 1, wherein the electromagnet circumscribes an inner surface of the base portion of the colpotomy cup.

4. The colpotomy system of claim 1, wherein the electromagnet circumscribes an exterior surface of the top portion of the colpotomy cup.

5. The colpotomy system of claim 1, wherein the electromagnet circumscribes an inner surface of the top portion of the colpotomy cup.

6. The colpotomy system of claim 1, wherein the top portion of the colpotomy cup extends along the longitudinal axis from a first surface to a second surface, wherein the first surface defines a leading edge and the second surface couples to the base portion of the colpotomy cup.

7. The colpotomy system of claim 6, wherein the first diameter is selected from a range of 3 cm to 4 cm.

8. The colpotomy system of claim 1, wherein the electromagnet comprises a solenoid.

9. The colpotomy system of claim 8, further comprising a ferromagnetic core arranged within an inner volume of the solenoid and circumscribed by the solenoid, wherein the ferromagnetic core augments the magnetic field generated by the solenoid.

10. The colpotomy system of claim 8, wherein the solenoid is a first solenoid and the electromagnet further comprises a second solenoid arranged within an inner volume of the first solenoid and circumscribed by the first solenoid.

11. The colpotomy system of claim 10, wherein the first solenoid and the second solenoid are coaxial.

12. The colpotomy system of claim 10, wherein when a first current passes through the first solenoid a first magnetic field is generated along a first direction thereby generating a first Lorentz force directed inward towards the longitudinal axis of the colpotomy cup, and when a second current passes through the second solenoid a second magnetic field is generated along a second direction that is opposite the first direction thereby generating a second Lorentz force directed outward from the longitudinal axis of the colpotomy cup.

13. The colpotomy system of claim 1, further comprising a bipolar electrocautery electrode having an electrical lead portion extending along a first direction and a cutting edge portion extending along a second direction that is perpendicular to the first direction.

14. The colpotomy system of claim 13, wherein the electrical lead portion and the cutting edge portion comprise a continuous conductor that extends along the first direction from a starting point in the electrical lead portion to the cutting edge portion where the continuous conductor extends along the second direction before returning along the first direction to an ending point in the electrical lead portion.

15. The colpotomy system of claim 14, wherein the continuous conductor defines a T-shaped conductive path.

16. A colpotomy kit, comprising:
a colpotomy cup having a top portion of a first diameter and a base portion of a second diameter that is smaller than the first diameter, wherein the base portion has a hole formed therein, wherein the hole circumscribes a longitudinal axis extending from the base portion to the top portion;
a solenoid coupled to at least one of the top portion or the base portion of the colpotomy cup; and
a bipolar electrocautery wand having an electrical lead portion extending along a first direction and a cutting edge portion extending along a second direction that is perpendicular to the first direction;
wherein when a current passes through the solenoid a magnetic field is generated along the longitudinal axis of the colpotomy cup thereby generating a Lorentz force directed inward towards the longitudinal axis of the colpotomy cup; and
wherein when a current passes through the cutting edge portion of the electrocautery wand, the Lorentz force pushes the cutting edge portion of the electrocautery wand towards the longitudinal axis of the colpotomy cup.

17. The colpotomy kit of claim 16, wherein the solenoid circumscribes an exterior surface of the base portion of the colpotomy cup.

18. The colpotomy kit of claim 16, wherein the solenoid circumscribes an inner surface of the base portion of the colpotomy cup.

19. The colpotomy kit of claim 16, wherein the solenoid circumscribes an exterior surface of the top portion of the colpotomy cup.

20. The colpotomy kit of claim 16, wherein the solenoid circumscribes an inner surface of the top portion of the colpotomy cup.

21. The colpotomy kit of claim 16, wherein the solenoid is a first solenoid and the colpotomy kit further comprises a second solenoid arranged within an inner volume of the first solenoid and circumscribed by the first solenoid.

22. The colpotomy kit of claim 21, wherein when a first current passes through the first solenoid a first magnetic field is generated along a first direction thereby generating a first Lorentz force directed inward towards the longitudinal axis of the colpotomy cup, and when a second current passes through the second solenoid a second magnetic field is generated along a second direction that is opposite the first direction thereby generating a second Lorentz force directed outward from the longitudinal axis of the colpotomy cup.
